(19) United States
(12) Patent Application Publication
(10) Pub. No.: US 2003/0017521 A1
(43) Pub. Date: Jan. 23, 2003

(54) Identification of antimicrobial agents

(75) Inventors: Debabrata RayChaudhuri, Somerville, MA (US); Danielle Margalit, Brookline, MA (US); Marc Kirschner, Newton, MA (US)

Correspondence Address: Choate, Hall & Stewart, Exchange Place, 53 State Street, Boston, MA, 02109, US

(73) Assignee: President and Fellows of Harvard College

(21) Appl. No.: 10/153,268

(22) Filed: May 22, 2002

Related U.S. Application Data
(60) Provisional application No. 60/292,883, filed on May 22, 2001.

(51) Int. Cl. C12Q 1/48; A61K 31/00

(52) U.S. Cl. 435/15; 514/1

(57) ABSTRACT

The present invention provides novel assay systems and methods of using these assays systems for identifying compounds that affect microbial cell division. The present invention further provides pharmaceutical compositions that have anti-microbial activity and methods of treating microbial infections.

### PRIORITY INFORMATION

This application is related to the subject matter in and claims benefit of pending U.S. provisional application Serial No. 60/292,883, filed May 22, 2001, the entire contents of which are incorporated herein by reference.

### GOVERNMENT SUPPORT

Development of the present invention was funded by a grant from the Department of Defense Advanced Research Projects Agency(Grant Number N65236-98-1-5408. Accordingly, the United States Government may have certain rights in the invention.

### BACKGROUND OF THE INVENTION

Anti-microbial agents, such as antibiotics, have been effective tools in the treatment of infectious diseases during the last half century. The systematic screening of natural product libraries from soil samples or marine environments has generated most of the classes of anti-bacterial agents used today (e.g., β-lactams, aminoglycosides, macrolides, and sulfonamides, to name a few). Additionally, these initial leads have, in many cases, been subsequently modified to produce second and third generation therapeutics with one or more of broadened anti-microbial activity, enhanced oral bioavailability, and improved toxicological and pharmacokinetic properties.

From the time that antibiotic therapy was first developed to the late 1980s, there was almost complete control over bacterial infections in developed countries. However, the emergence of resistant bacteria, especially during the late 1980s and early 1990s, is changing this situation (see, for example, Breithaupt, H., “The New Antibiotics: Can Novel Anti-bacterial Treatments Combat the Rising Tide of Drug-Resistant Infections?” Nature Biotechnology, (1997) 17: 1165). The increase in antibiotic resistant strains has been particularly common in major hospitals and care centers. The consequences of the increase in resistant strains include higher morbidity and mortality, longer patient hospitalization, and an increase in treatment costs. (B. Murray, New Engl. J Med. 330: 1229-1230 (1994)).

One major factor that is contributing to the increase in the number of resistance strains is the over-use and/or inappropriate administration of anti-microbials in the treatment arena. Newly acquired resistance is generally due to the relatively rapid mutation rate in bacteria. Another contributing factor is the ability of many microorganisms to exchange genetic material that confers resistance, e.g., exchanging of resistance plasmids (R plasmids) or resistance transposons.

For example, following years of use to treat various infections and diseases, penicillin resistance has become increasingly widespread in the microbial populations that were previously susceptible to the action of this drug. Some microorganisms produce β-lactamase, an enzyme that destroys the anti-microbial itself, while other microorganisms have undergone genetic changes that result in alterations to the cell receptors known as the penicillin-binding proteins, such that penicillin no longer effectively binds to the receptors. Other organisms have evolved in a manner that prevents the lysis of cells to which the drug has bound. The drug therefore inhibits the growth of the cell, but does not kill the cell. This appears to contribute to the relapse of disease following premature discontinuation of treatment, as some of the cells remain viable and may begin growing once the anti-microbial is removed from their environment.

The first report of penicillin resistance occurred in Australia in 1967. Since this initial report, increasing numbers of penicillin resistant strains have been reported worldwide. In addition, strains having resistance to numerous other antibiotics have also been reported, including strains that are resistant to chloramphenicol, erythromycin, tetracycline, clindamycin, rifampin, and sulfamethoxazole-trimethoprim.

Microorganisms that are resistant to this wide range of drugs include opportunistic and virulent pathogens that were previously susceptible to antibiotic treatment. Resistant opportunistic pathogens are problematic for debilitated or immunocompromised patients, while the development of tolerance and resistance in virulent pathogens poses a significant threat to the ability to treat disease in all patients, compromised and non-compromised. Infections resulting from these naturally resistant opportunistic or virulent pathogens are becoming more difficult to treat with currently available antibiotics.

Clearly, in order to maintain the standard of public health we enjoy today, there is an urgent medical need for the identification of compounds having anti-microbial activity that can override existing mechanisms of resistance. Preferably, the anti-microbial compounds are active against a broad spectrum of microorganisms, while remaining non-toxic to human and other mammalian cells.

### SUMMARY OF THE INVENTION

The invention provides assay systems and methods of using these assay systems for screening compounds for anti-microbial activity, and more particularly, to using bacterial proteins in vitro to detect compounds that interfere with cell division. For example, the present invention provides cell-free assays to screen compounds for their anti-microbial activity that utilizes bacterial proteins. In another embodiment, the present provides cell-based assays that utilize conditional-lethal bacterial mutants in target gene products to screen compounds for anti-microbial activity.

The present invention further provides pharmaceutical compositions including anti-microbial agents and method of using such pharmaceutical compositions to treat microbial infections and/or disorders related to microbial infections. The compounds can be used in combination with other agents for the prophylaxis and treatment of conditions associated with microbial infections and/or disorders related to microbial infections.

In certain preferred embodiments, microorganisms are not resistant to the identified anti-microbial agents, exhibit improved bioavailability, and/or have minimal side effects. In a particularly preferred embodiment of the invention the compounds are effective against certain microorganisms that are resistant to some or even all of the anti-bacterial agents that are currently approved or in clinical trials.

The pharmaceutical compositions can be used alone or in combination with other agents for the prophylaxis and treatment of conditions associated with microbial infections or disorders related to microbial infections. In general, the inventive compositions comprise an effective amount of an anti-microbial compound or a pharmaceutically acceptable salt thereof, in combination with a pharmaceutically acceptable carrier, such as a diluent or excipient.

In still another aspect, the invention provides methods for prophylaxis and/or treatment of conditions associated with microbial infections and/or disorders related to microbial infections by administering an effective amount of an inventive compound. In particular, the invention provides a method for the treatment or prophylaxis of conditions associated with microbial infections and/or disorders related to microbial infections comprising administering to a host (such as a bird, fish, or cell) or patient, such as a primate, an effective amount of a compound of the present invention.

In certain preferred embodiments combination therapies are provided wherein an effective amount of a compound of the present invention, and an effective amount of one or more other compounds useful in the treatment of conditions associated with microbial infections and/or disorders related to microbial infections, are administered to a host or patient.

In yet another aspect, the present invention also provides pharmaceutical packs or kits comprising one or more containers filled with one or more of the ingredients of the pharmaceutical compositions of the invention, and in certain embodiments, include an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration. The invention further provides novel assays for the identification of agents having anti-microbial activity, e.g., anti-bacterial, e.g., any eubacteria or archaebacteria. In particular, these assays inhibit the ubiquitous prokaryotic cell division protein FtsZ. Such anti-microbial agents have the activity of inhibiting cell division by blocking the formation of the FtsZ ring that is crucial for septation. In other embodiments, the identified compounds and compositions may be inhibitory to plant cell division and be useful to kill weeds.

### BRIEF DESCRIPTION OF THE DRAWING

The invention is described with reference to the several figures of the drawing.

FIG. 1 shows a diagram of a FtsZ ring structure and photographs of a FtsZ ring structure by immunofluorescence in E. coil cells.

FIG. 2 is an illustration that depicts an overview of a screening process for identifying FtsZ inhibitors or enhancers.

FIG. 3 illustrates the chemical structure of various inhibitors of FtsZ activity.

FIG. 4 is an electron micrograph image of the effect of compounds 18M04 and 27D12 that destabilize FtsZ polymers in a dose dependent manner.

FIG. 5 is an electron micrograph image of the effect of compounds 16L-09, 27F02, and 58P18 that cause mild bundling of FtsZ protofilaments.

FIG. 6 illustrates the chemical structure of an inhibitor of FtsZ ring assembly.

FIG. 7 illustrates the in vitro enzyme-coupled assay for assembly dependent FtsZ GTPase activity.

FIG. 8 is a flow chart that depicts the in vitro FtsZ screen that was carried out to identify five inhibitors of FtsZ activity.

FIG. 9 is a photograph of DRC39 E coli cells immunostained for the FtsZ ring assembly after 60 minutes treatment with the inhibitor 26E-10.

FIG. 10 is a photograph of DRC39 E coli cells immunostained for the FtsZ ring assembly after 90 minutes treatment with the inhibitor 26E-10.

FIG. 11 is a table showing minimum inhibitory concentrations of compounds on growth of E. coli (WT), E. coli (acrAB), E. coli ftsZ84acr AB, and Vibrio cholera.

FIG. 12 depicts the percent FtsZ inhibition of compounds from the MDS1 (galanthamine) library measured by the NADH assay.

FIG. 13 is a photograph of DRC39 cells, which are wild-type E. coli cells that have a knockout of the multidrug efflux pump AcrAB) treated for two hours with 27F02.

FIG. 14 shows photographs showing the sensitivity of DRC40/pBR322 mutant E. coli cells to the inhibitor 26E-10 (panel A) and the sensitivity of the ftsZ84 mutant DRC40 carrying pBR-ftsZ⁺ (panel B).

FIG. 15 shows photographs of the effect of 58P-18 on FtsZ ring assembly in DRC39 cells.

FIG. 16 shows photographs of the FtsZ ring structure of ftsZ84 mutants at permissive and non-permissive temperatures.

Definitions

As discussed above, the present invention provides pharmaceutical compositions including compounds useful in the eradication or inactivation (i.e., affect their inability to replicate) of harmful microorganisms prior to infection and thus can be utilized as preventative and/or disinfectant agents.

It will be appreciated by one of ordinary skill in the art that numerous asymmetric centers may exist in the compounds of the present invention. Thus, inventive compounds and pharmaceutical compositions thereof may be in the form of an individual enantiomer, diastereomer or geometric isomer, or may be in the form of a mixture of stereoisomers.

Additionally, the present invention provides pharmaceutically acceptable derivatives of the foregoing compounds, and methods of treating animals using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety that is susceptible to removal in vivo yielding the parent molecule as the pharmacologically active species. An example of a pro-drug is an ester that is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment of and/or prevention of bacterial infections, protozoal infections, or for disorders related to microbial infections. The term “stable”, as used herein, preferably refers to compounds which possess stability sufficient to allow manufacture and which maintain the integrity of the compound for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include alkyl, alkenyl, alkynyl, cycloalkyl, cycloalkenyl, and cycloalkynyl moieties. Thus, as used herein, the term “alkyl” includes both straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups.

Unless otherwise specified, alkyl and other aliphatic groups preferably contain 1-6, or 1-3, contiguous aliphatic carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents.

In certain embodiments of the present invention C₁-C₃ or C₁-C₆ alkyl moieties are employed. As used herein, the terms “C₁-C₃-alkyl” and “C₁-C₆-alkyl” refer to saturated, substituted or unsubstituted, straight- or branched-chain hydrocarbon radicals derived from a hydrocarbon moiety containing between one and three, and one and six carbon atoms, respectively, by removal of a single hydrogen atom. Examples of C₁-C₃-alkyl radicals include, but are not limited to, methyl, ethyl, propyl and isopropyl. Examples of C₁-C₆-alkyl radicals include, but are not limited to, methyl, ethyl, propyl, isopropyl, n-butyl, tert-butyl, neopentyl and n-hexyl.

In certain embodiments of the present invention, C₂-C₆ alkenyl moieties are employed. The term “C₂-C₆-alkenyl” denotes a monovalent group derived from a hydrocarbon moiety containing from two to six carbon atoms and having at least one carbon-carbon double bond by the removal of a single hydrogen atom. Additionally, the C₂-C₆ alkenyl moieties, as used herein, may be substituted or unsubstituted. Alkenyl groups include, but are not limited to, for example, ethenyl, propenyl, butenyl, 1-methyl-2-buten-1-yl, and the like.

In certain embodiments of the present invention, C₂-C₆ alkynyl moieties are employed. The term “C₁-C₆-alkynyl” as used herein refers to a monovalent group derived from a hydrocarbon containing from two to six carbon atoms and having at least one carbon-carbon triple bond by the removal of a single hydrogen atom. Additionally, the C₂-C₆ alkenyl moieties, as used herein, may be substituted or unsubstituted. Representative alkynyl groups include, but are not limited to, ethynyl, 2-propynyl (propargyl), 1-propynyl and the like.

The term “C₁-C₆-alkoxy” as used herein refers to a C₁-C₆-alkyl group, as previously defined, attached to the parent molecular moiety through an oxygen atom. Examples of C₁-C₆-alkoxy, but are not limited to, methoxy, ethoxy, propoxy, isopropoxy, n-butoxy, tert-butoxy, neopentoxy and n-hexoxy.

The term “alkylamino” refers to a group having the structure —NHR′ wherein R′ is alkyl, as defined herein. Examples of alkylamino include, but are not limited to, methylamino, ethylamino, iso-propylamino and the like. In certain embodiments, C₁-C₃ alkylamino groups are utilized in the present invention. The term “C₁-C₃-alkylamino” as used herein refers to one or two C₁-C₃-alkyl groups, as previously defined, attached to the parent molecular moiety through a nitrogen atom. Examples of C₁-C₃-alkylamino include, but are not limited to methylamino, dimethylamino, ethylamino, diethylamino, and propylamino.

Some examples of substituents of the above-described aliphatic (and other) moieties of compounds of the invention include, but are not limited to: F, Cl, Br, I, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCONH-heteroaryl, SO₂-C₁-C₆-alkyl, SO₂-aryl, C₃-C₆-cycloalkyl, CF₃, CH₂CF₃, CHCl₂, CH₂OH, CH₂CH₂OH, CH₂NH₂, CH₂SO₂CH₃, aryl, heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkyl-amino, thio, aryl-thio, heteroarylthio, benzyl-thio, C₁-C₆-alkyl-thio, or methylthiomethyl. Additional examples of generally applicable substituents are illustrated by the specific embodiments shown in the Examples that are described herein.

The term “aprotic solvent” as used herein refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heteroaryl compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. 11, in the Techniques of Chemistry Series, John Wiley & Sons, NY, 1986. In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted.

Substituents include, but are not limited to, any of the previously mentioned substituents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiments of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl,oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one, two or three of the hydrogen atoms thereon independently with any one or more of the following moieties including, but not limited to: F, Cl, Br, I, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCONH-heteroaryl, SO₂-C₁-C₆-alkyl, SO₂-aryl, C₃-C₆-cycloalkyl, CF₃, CH₂CF₃, CHCl₂, CH₂OH, CH₂CH₂OH, CH₂NH₂, CH₂SO₂CH₃, aryl, heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkyl-amino, thio, aryl-thio, heteroarylthio, benzyl-thio, C₁-C₆-alkyl-thio, or methylthiomethyl. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted. F, Cl, Br, I, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH-C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH-C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCONH-heteroaryl, SO₂—C₁-C₆-alkyl, SO₂-aryl, C₃-C₆-cycloalkyl, CF₃, CH₂CF₃, CHCl₂, CH₂OH, CH₂CH₂OH, CH₂NH₂, CH₂SO₂CH₃, aryl, heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkyl-amino, thio, aryl-thio, heteroarylthio, benzyl-thio, C₁-C₆-alkyl-thio, or methylthiomethyl. Additional examples of generally applicable substituents are illustrated by the specific embodiments shown in the Examples that are described herein.

The term “heteroaliphatic”, as used herein, refers to aliphatic moieties which contain one or more oxygen, sulfur, nitrogen, phosphorous or silicon atoms, e.g., in place of carbon atoms. Heteroaliphatic moieties may be branched, unbranched or cyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to: F, Cl, Br, I, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCONH-heteroaryl, SO₂—C₁-C₆-alkyl, SO₂-aryl, C₃-C₆-cycloalkyl, CF₃, CH₂CF₃, CHCl₂, CH₂OH, CH₂CH₂OH, CH₂NH₂, CH₂SO₂CH₃, aryl, heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkyl-amino, thio, aryl-thio, heteroarylthio, benzyl-thio, C₁-C₆-alkyl-thio, or methylthiomethyl. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl”, as used herein, refers to a non-aromatic 5-, 6- or 7-membered ring or a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl” group is utilized and as used herein, refers to a heterocycloalkyl group, as defined above, substituted by independent replacement of one, two or three of the hydrogen atoms thereon with but are not limited to: F, Cl, Br, I, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCONH-heteroaryl, SO₂—C₁-C₆-alkyl, SO₂-aryl, C₃-C₆-cycloalkyl, CF₃, CH₂CF₃, CHCl₂, CH₂OH, CH₂CH₂OH, CH₂NH₂, CH₂SO₂CH₃, aryl, heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkyl-amino, thio, aryl-thio, heteroarylthio, benzyl-thio, C₁-C₆-alkyl-thio, or methylthiomethyl. Additional examples of generally applicable substituents are illustrated by the specific embodiments shown in the Examples that are described herein.

“Hydroxy-protecting group”, as used herein, refers to an easily removable group, which is known in the art to protect a hydroxyl group against undesirable reaction during synthetic procedures and to be selectively removable. The use of hydroxy-protecting groups is well known in the art for protecting groups against undesirable reactions during a synthetic procedure and many such protecting groups are known, cf., for example, T. H. Greene and P. G. M. Wuts, Protective Groups in Organic Synthesis, 2^{nd} edition, John Wiley & Sons, New York (1991). Examples of hydroxy-protecting groups include, but are not limited to, methylthiomethyl, tert-dimethylsilyl, tert-butyldiphenylsilyl, ethers such as methoxymethyl, and esters including acetyl benzoyl, and the like.

The term “oxo” denotes a group wherein two hydrogen atoms on a single carbon atom in an alkyl group as defined above are replaced with a single oxygen atom (i.e. a carbonyl group).

The term “protected-hydroxy” refers to a hydroxy group protected with a hydroxy protecting group, as defined above, including benzoyl, acetyl, trimethylsilyl, triethylsilyl, methoxymethyl groups, for example.

The term “protogenic organic solvent” as used herein refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4^{th} ed., edited by John A. Riddick et al., Vol. 11, in the Techniques of Chemistry Series, John Wiley & Sons, NY, 1986.

The term “treating”, as used herein, unless otherwise indicated, means reversing, alleviating, inhibiting the progress of, or preventing the disorder or condition to which such term applies, or one or more symptoms of such disorder or condition. Them term “treatment”, as used herein, refers to the act of treating, as “treating” is defined immediately above.

DRC39 is the MC 1000 (ftsZ⁺) delta acrAB::kan strain of E. coli.

DRC40 is the DRC13 (ftsZ84) delta acrAB::kan strain of E. coli.

### DETAILED DESCRIPTION OF CERTAIN PREFERRED EMBODIMENTS

As discussed above, the invention relates to assay systems and the uses of these assay systems for screening compounds for anti-microbial activity, and more particularly, to using bacterial proteins in vitro to detect compounds that interfere with cell division. In one embodiment, the present invention provides cell-free assays to screen compounds for their anti-microbial activity that utilize bacterial proteins. In another embodiment, the present provides in vivo cellular assays that utilize bacterial proteins to screen compounds for anti-microbial activity.

The present invention further relates to pharmaceutical compositions including compounds useful in the treatment and/or prevention of one or more microbial infections. Those skilled in the art will appreciate that this includes compounds that inhibit the growth of microbial cells, such as yeast, fungi, protozoa, bacteria, and the like.

Assay Systems and Methods of Use

Bacterial cells divide by first initiating DNA replication. At the end of the bacterial cell cycle, the chromosomes segregate and the cells divide by forming a septum that divides the cells in two, a process known as septation.

A large collection of mutants that block DNA replication and/or cell division have been identified in a wide range of microbial cells. In many cases, the gene(s) responsible for the mutant phenotypes and their wild-type counterparts have been cloned and characterized. The in vivo and in vitro activities of such wild-type and mutant proteins may be employed to identify inhibitors of DNA replication and/or cell division and thus identify inhibitors of microbial cell growth. Furthermore, a protein that is a key player in one type of microbial cell, for example, a bacterial cell, may be conserved in another type of microbial cell, e.g., a fungal cell. Thus, inhibitors that block the activity of these proteins to prevent cell division might also overlap between different microbial cell types.

Such anti-microbial agents may be used as broad spectrum therapeutics, e.g., as anti-microbial agents. Alternatively, such anti-microbial agents may be used for decontamination, e.g., decontamination of water having a high microbial count. It may also be appreciated that molecules that activate the activity of a protein involved in the cell cycle may also be identified, which may spur further basic research.

One protein that participates in bacterial cell division is the FtsZ protein. FtsZ is essential for bacterial cell multiplication and is ubiquitous in the prokaryotic kingdom, being present in eubacteria (gram-positive/gram-negative), archaea, mycoplasmas, chloroplasts, and mitochondria of lower eukaryotes), while it is absent from the mitochondria of higher eukaryotes (yeast to humans). It is also absent from the obligate intracellular bacterial pathogen, Clamydia trachomatis. Therefore, because inhibitors of FtsZ activity are expected to block cell division in a wide range of prokaryotic organisms, molecules that modulate FtsZ function may be developed as broad spectrum anti-bacterial agents against known and unknown bacterial pathogens.

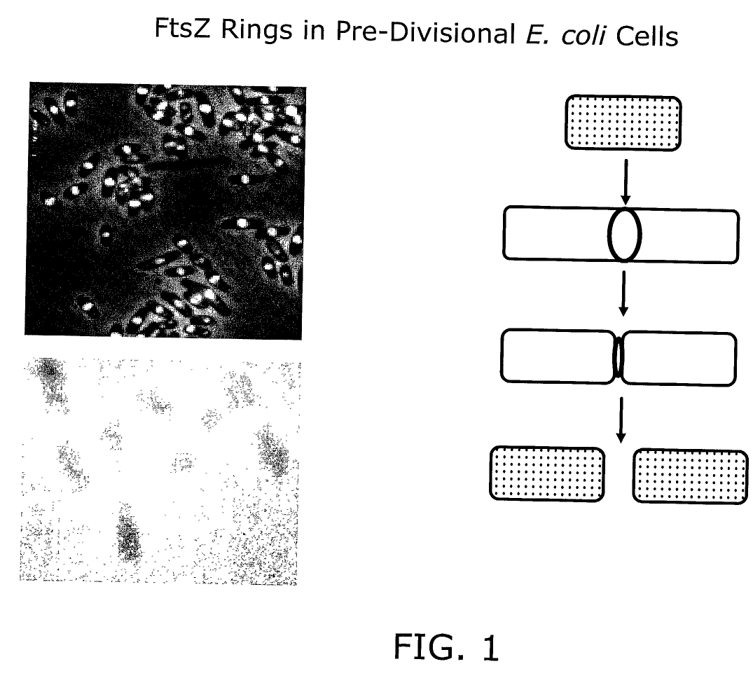

FtsZ is a tubulin-like GTPase that forms a membrane-associated cytokinetic contractile ring structure in vivo at the site of division in bacterial cells (see FIG. 1, which shows localization of FtsZ at the cytokinetic ring structure in predivisional E. coli cells). During the process of cell division, FtsZ becomes concentrated at the inner membrane into a ring-like structure at the prospective division site immediately before the start of cell division. During septation, the diameter of the FtsZ ring (also referred to herein as the Z ring) becomes progressively smaller as it remains at the leading edge of the invaginating cell wall.

FtsZ is believed to interact with several different molecules that also play specific roles in one or more cell division processes. Genetic studies have suggested possible interactions between FtsZ and several other proteins. For example, FtzZ has been shown to interact with FtsA by yeast two-hybrid analysis and by the ability of the FtsZ ring to recruit FtsA. Indeed FtsA can be co-purified with FtsZ and vice-versa. FtsZ also is known to interact with ZipA, a protein essential for cell viability. Cells lacking sufficient ZipA activity die. Thus, those skilled in the art will appreciate that large screens for compounds that either inhibit or activate the ability of FtsZ to interact with FtsA or ZipA have great flexibility in their design and implementation.

In vitro, FtsZ polymerizes in a guanine nucleotide-dependent manner into structures (protofilaments or protofilament bundles or sheets) that are similar to tubulin polymers (microtubules). These activities, or more particularly, the inhibition or activation of these activities, may be used to identify test compounds, such as peptide and small molecule compounds that are inhibitors or activators of FtsZ-mediated cell division.

The likelihood of prokaryotic cells developing resistance to molecules that inhibit FtsZ is relatively low for several reasons. First, FtsZ orthologs have a high degree of sequence conservation, especially in domains involved in GTP binding and hydrolysis, in subunit interaction required for polymerization, and in the interaction with proteins such as FtsA and ZipA. Second, as demonstrated herein in Example 1, low, sub-stoichiometric levels of FtsZ inhibitors are likely to be required to affect FtsZ function. Third, and most importantly, FtsZ is an essential, non-redundant protein, required for cell division. Use of FtsZ inhibitors may further provide an advantage when used in combination with other drug treatments in that it may provide a valuable time window for other drug treatments to have an effect by slowing down the rate of multiplication of the infectious organism.

In preferred embodiments, the present invention provides methods of identifying compounds that are inhibitors or activators of FtsZ activity (the first anti-microbial compounds identified to target a bacterial cell division protein). In related embodiments, the present invention provides methods of identifying compounds that are inhibitors or activators of proteins that interact with FtsZ, such as FtsA and ZipA.

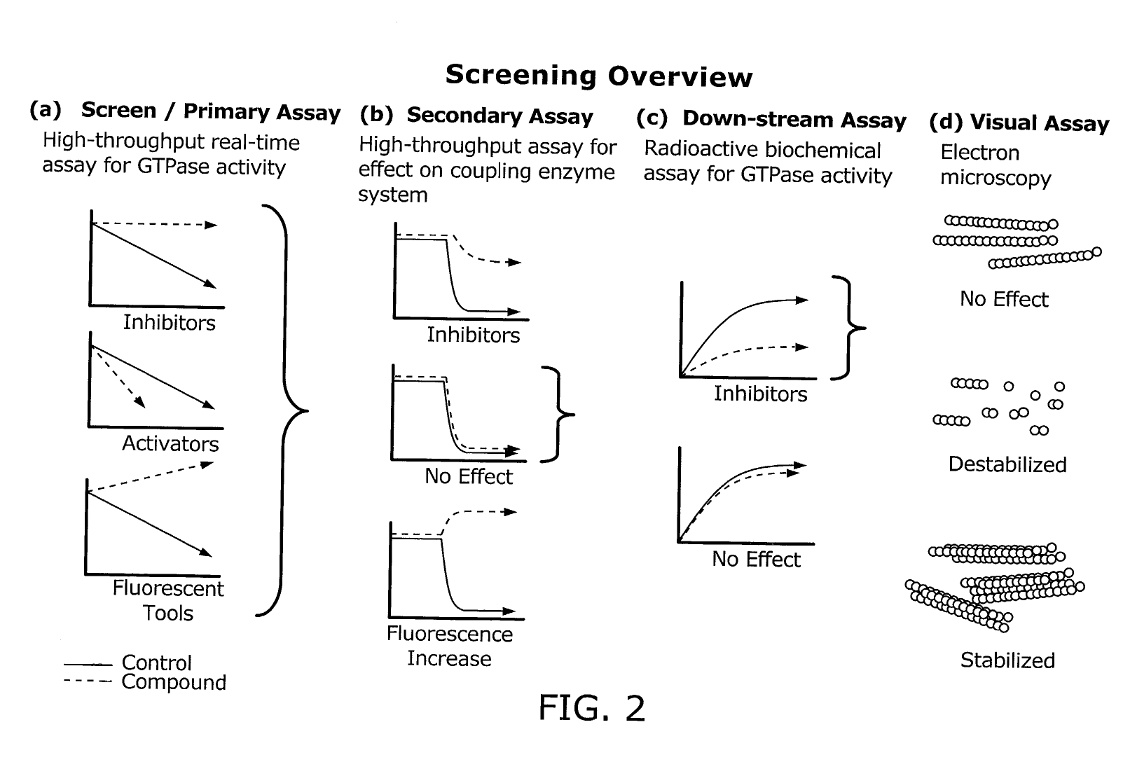

In one preferred embodiment, the present invention provides real-time, enzyme coupled assays for FtsZ GTPase activity that are amenable to miniaturization for high-throughput screening (see FIG. 2, panel A). According to certain preferred embodiments, the real time assay can be used as a primary screening assay for FtsZ inhibitors or activators. A secondary assay, such as a high-throughput assay that measures the effect of the compound on the coupling enzyme system may be used to verify the results of the real time assay used in the primary screen (FIG. 2, panel B). Once the results are verified, a down-stream assay may be used to determine interacting proteins (FIG. 2, panel C). A visual assay may further be used to assess the stabilizing or destabilizing effects of the agent (FIG. 2, panel D).

In other preferred embodiments, the present invention provides assays for FtsZ activity that are based on cell morphology and FtsZ ring assembly in vivo in wild-type and ftsZ mutant cells. A visual assay may be used to determine the effect of a compound on polymerization, e.g., destabilizing or stabilizing polymerization (see FIG. 2, panel D). Other available assays include charcoal-based and thin-layer chromatographic assays for GTPase activity, negative-stain transmission electron microscopy to assess the activity of a compound on FtsZ polymers, and growth assays for assessing the anti-microbial activity of a compound. Such assays may include experiments that assess cell culture growth by, for example, culture turbidity in response to addition of compound.

For example, inhibition of FtsZ activity results in a block in the ability to form a cytokinetic ring structure, which results in abnormally long cells due to a decrease in septation without affecting cellular mass increase. An inhibition of FtsZ activity can also be measured in vitro by detecting a decrease in GTP-dependent polymerization of FtsZ and the concomitant GTPase activity. Alternatively, activation of FtsZ in vivo, or increased FtsZ abundance, results in hyper-formation of ring structures in the cell, which yields shortened cells due to polar septation. Similarly, an increase in in vitro polymerization-dependent GTPase activity may be observed in the presence of a FtsZ activator.

More particularly, the present invention provides methods of using the FtsZ protein and proteins that interact with FtsZ, to screen for compounds having anti-microbial activity. The assay utilizes FtsZ and/or FtsZ-associated bacterial proteins in vitro to detect compounds that interfere with cell division. In other embodiments, the present invention provides an in vivo cellular assay that utilizes FtsZ and/or FtsZ-associated proteins to screen compounds for anti-microbial activity.

More particularly, the present invention provides a real time assay system for measuring FtsZ activity. The assays system includes a reaction mixture having the following components: the enzymes FtsZ protein, pyruvate kinase, lactate dehydrogenase, and the substrates GTP, PEP, and NADH. Detection within the assay system involves measuring the rate of enzymatic conversion of NADH to NAD⁺ by lactate dehydrogenase by following fluorescence change. Miniaturization for high throughput screening may be achieved by adding the reagents (enzymes and substrates plus FtsZ) to a multi-well plate (e.g., a 384-well stock plate) using a robotic multipipetor, and measuring NADH fluorescence using, e.g., a Wallac Plate reader. All positive results may be tested against the coupling enzymes pyruvate kinase and lactate dehydrogenase to rule out the possibility of false positives.

It will be appreciated that the real time assay system described above may be used to screen any compound for an effect on FtsZ activity. Therefore, the present invention further provides a method of detecting compounds that affect (increases or decreases) FtsZ activity that involves combining purified FtsZ protein in a reaction mixture with the enzymes pyruvate kinase and lactate dehydrogenase, and the substrates GTP, PEP, and NADH and detecting an alteration in NADH fluorescence. More particularly, upon addition of FtsZ to the reaction mixture, FtsZ catalyzes a reaction with GTP yielding the products GDP and phosphate. One product of the FtsZ reaction, GDP, then becomes the substrate for pyruvate kinase with PEP yielding pyruvate and GTP. Pyruvate in turn becomes a substrate for lactate dehydrogenase with NADH to yield NAD⁺ and lactate. Inhibition or activation of FtsZ activity is determined by measuring the change in the rate of the decrease of NADH fluorescence compared to that obtained in the absence of the test molecule (excitation: 355 nm, emission: 460). Library compounds may be added to the reaction mixtures compared to reaction mixtures lacking any compounds to assess their effect on FtsZ activity.

A second assay system for identification of compounds affecting FtsZ activity includes a bacterial cell that has a mutation affecting a multidrug efflux pump and further includes an expression vector encoding the FtsZ protein. It will be appreciated that expression of proteins in bacteria is standard in the art, as demonstrated below (see also Sambrook et al., Molecular Cloning: A Laboratory Manual, Cold Spring Harbor Press, N.Y., Ausubel et al., Current Protocols in Molecular Biology, Greene Publishing Associates, New York, V. 1&2, 1996, each of which is incorporated by reference herein).

Thus, the present invention provides assays that detect the phenotype of wild-type and mutant bacterial cells (e.g., the congenic thermosensitive ftsZ84 E. coli mutant DRC13 and their derivatives, which lack the major multidrug efflux pump AcrAB) in the presence and absence of compound (see Example 1). In one preferred embodiment, the present invention provides an assay that involves the steps of 1) expressing the FtsZ protein in a wild-type cell, 2) contacting the cell with a compound, and 3) detecting a defect in cell division. For example, the defect in cell division may be an activation of cell division, e.g., caused by excessive intracellular polymerization of the FtsZ protein. This would result in a phenotype of excessively short cells without DNA, called minicells, resulting from division activity at the cell poles. In addition, under conditions of excessive intracellular polymerization of the FtsZ protein, the FtsZ rings would persist longer and more stably, thereby impeding ring constriction essential for septation. Alternatively, the defect in cell division may be an inhibition of cell division, e.g., caused by a blockage to intracellular polymerization of the FtsZ protein or hyperstabilization of the FtsZ polymers This block in FtsZ activity may result is long filamentous cells that divide infrequently or completely fail to divide.

In another embodiment, the compound is used in an assay that determines its ability to decrease or exacerbate a ftsZ phenotype. In certain preferred embodiments, the invention provides a method of identifying compounds that affect cell division, comprising steps of contacting a cell that is defective in cell division (e.g., ftsZ84 cells having a thermosensitive mutation in the gene encoding FtsZ) with a compound of interest; and detecting an alteration in the phenotype of the cell. More particularly, the method involves the steps of 1) contacting a ftsZ cell with a compound of interest, and 2) detecting an alteration in the phenotype of the ftsZ cell (e.g., a destabilization in the Z ring structure). Typically such ftsZ cells are temperature sensitive ftsZ cells, e.g., ftsZ84 cell that grow and divide at 30° C. and undergo a division block at 42° C. due to a destabilization of the Z-rings at high temperature. ftsZ84 cells lacking the multidrug efflux pump AcrAB would have a decreased ability to expel compound from the cell, thus allowing increased concentrations of a compound to accumulate in the cell potentially resulting in an exacerbated phenotype caused by the compound in that cell at the permissive temperature of 30° C. The phenotype in the presence of a compound that inhibits FtsZ activity would be an exacerbation of the failure to form Z rings in the ftsZ84 cells (a phenotype akin to the synthetic lethal interactions between two genetic mutations).

A third assay system provided by the present invention that may be utilized to identify compounds that affect cell division includes a bacterial cell that has a mutation affecting a multidrug efflux pump and further contains an expression construct encoding the ZipA protein. As noted above, as but one example of a cell that has a mutation affecting a multidrug efflux pump is the bacterial cell strain ftsZ84. In addition, as noted herein, the ZipA protein stabilizes intracellular assembly of the FtsZ ring. It has been shown that ftsZ84 cells expressing increased concentrations of ZipA, e.g., via introduction of a second copy of a ZipA gene into the cell, have decreased thermosensitivity at the restrictive temperature of 42° C.

In related embodiments, the present invention provides a method of utilizing the ftsZ84 strain in combination with a multidrug efflux pump mutation and a second copy ZipA, described above, to identify a compound that affects cell division, or alternatively a method of validating whether a compound affects cell division. The method involves observing the effect of the compound on the phenotype of ftsZ84 cells expressing increased concentrations of ZipA (see U.S. Pat. No. 5,948,889, incorporated by reference herein). Compounds that are inhibitors will diminish the suppression of the thermosensitivity of the ZipA expressing ftsZ84 cells at increased temperatures, resulting in a destabilization in the ring structure. Alternatively, a second copy of ZipA may increase the stability of the FtsZ ring in ftsZ84 cells and may thereby alleviate the lethality of FtsZ compounds.

Alternatively, or additionally, other assay systems may be used to identify compounds that affect cell division that measure the effect of a compound on FtsZ activity. One such assay system is the charcoal-based GTPase assay described by Lee et al. J. Biol. Chem. 267:1212-1218 (1992), incorporated herein by reference. Another assay is the malachite green-phosphomolybdate assay are shown below (Akiyama, Y., Kihara, A., Tokuda, H. and Ito, K. 1996, J. Biol. Chem. 271:31196-31201, incorporated herein by reference. Yet another assay includes negative-strain transmission electron microscopy of FtsZ polymers. More traditional anti-microbial screening assays are described by de Boer et al. in U.S. Pat. No. 5,948,889 (col. 8-9), incorporated herein by reference.

It will be appreciated that any compound may be tested on any assay system described herein to detect activators or inhibitors of cell division. Furthermore, any compound may be tested on any art available system that measures cell division. It will be appreciated that such compounds may be generated by any art available means. For example, the compounds of the galanthamine library, described in U.S. patent application Ser. No. 09/863,141, incorporated herein by reference in its entirety.

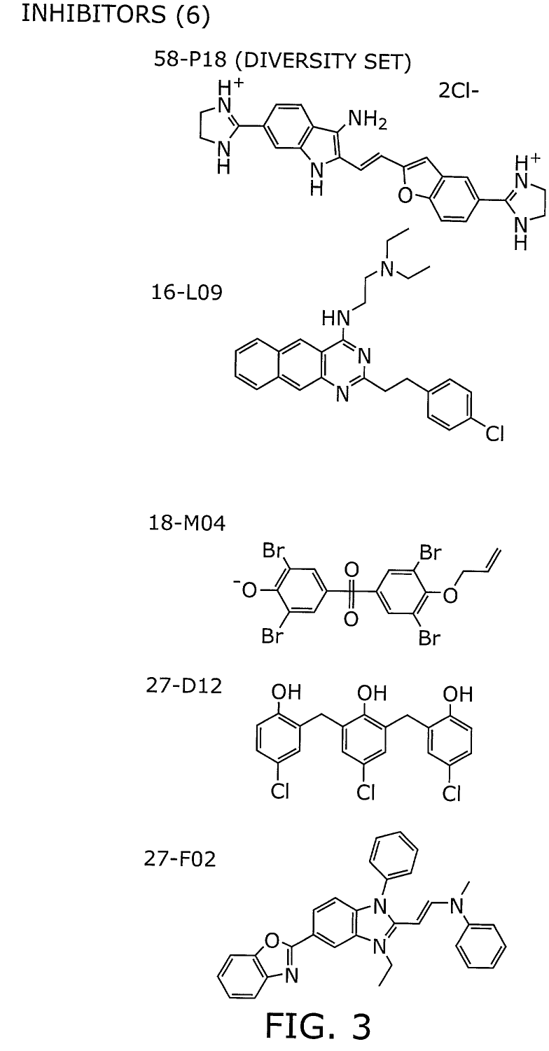

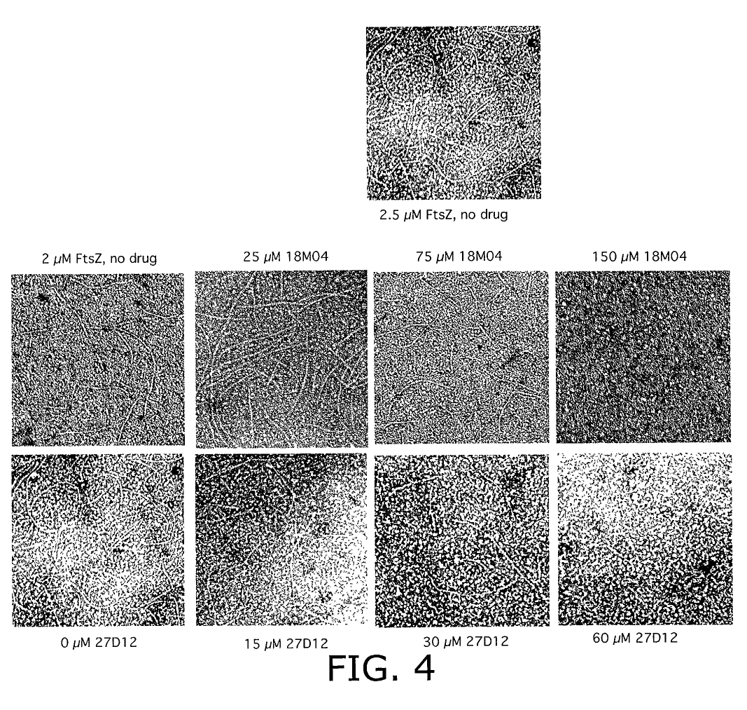

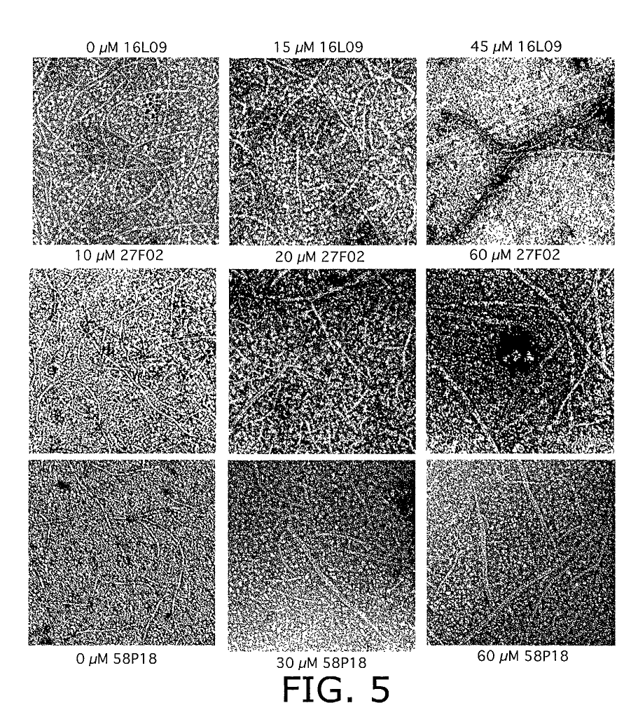

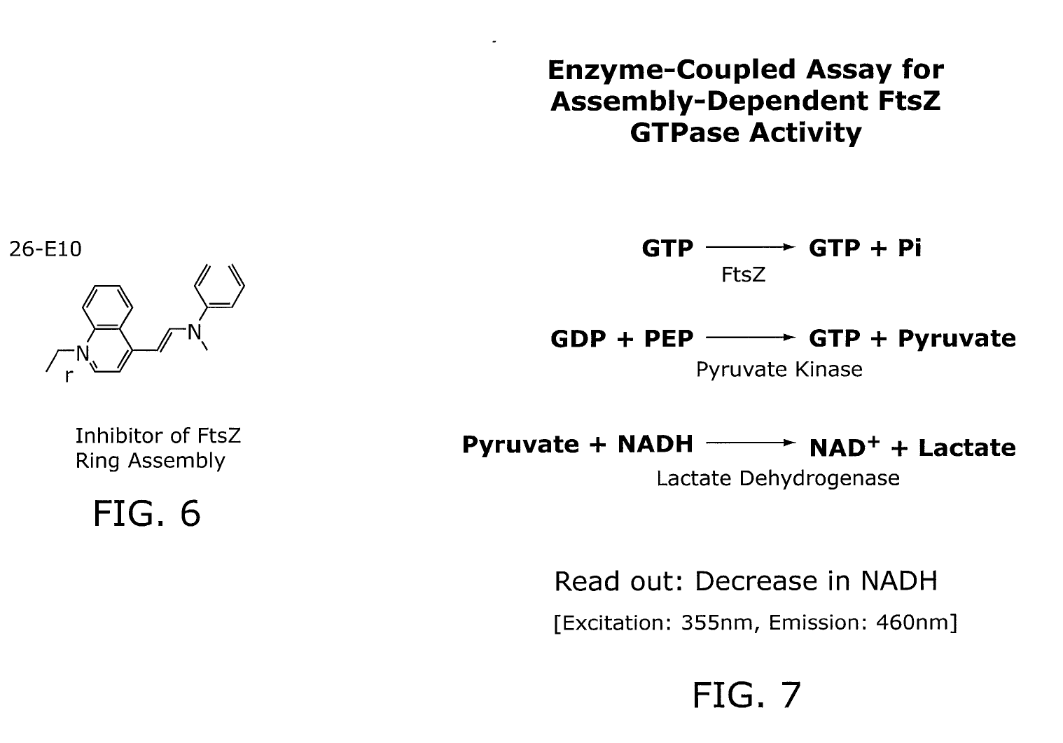

Two libraries (Chembridge Library and the NCI Diversity Library) were screened for molecules that inhibit or activate FtsZ activity as described herein (see Examples below). Out of a total of approximately ˜18,320 molecules, five inhibitors were identified and later verified in various in vivo and in vitro assays for bacterial growth and the formation of the FtsZ ring structure in the cell. The five of the inhibitors include 58P-18, 16L-09,18M-04, 27D-12, and 27F-02, which are depicted in FIG. 3. These compounds can be divided into two classes. The first class includes the compounds 18M04 and 27D12 that have a dose dependent destabilizing effect on the polymers (see FIG. 4). The second class of compounds includes 16L-09. 27F02, and 58P18, which cause mild bundling of FtsZ protofilaments (mostly via pairing of protofilaments) that could alter the FtsZ ring dynamics in vivo (FIG. 5). One compound, 26E-10, was identified in a cell based assay and is depicted in FIG. 6.

The IC50 values for some of these compounds are shown below.

TABLE 1
IC50 values against M. tuberculosis for FtsZ.

| | IC50 (μM) against |
|---|---|
| Compound | M. tub. FtsZ |
| 58-P18 | ˜30-35 |
| 27-F02 | ˜60 |
| 16-L09 | ˜50 |
| 27-D12 | ˜55 |

IC50 values obtained using the malachite green-phosphomolybdate assay are shown (Akiyama, Y., Kihara, A., Tokuda, H. and Ito, K. 1996, J. Biol. Chem. 271:31196-31201, incorporated herein by reference).

A subset of the inhibitors could kill bacterial cells at surprisingly low concentrations, ranging from 2-25 μg/ml. Two compounds, 26E-10 and 58-P18, appeared to affect cell division by targeting FtsZ ring formation in vivo. The compound 26E-10 has a strong in vivo phenotype, as demonstrated below. Moreover, 58P-18 shows both in vitro inhibition and an in vivo phenotype.

Pharmaceutical Compositions

As described above, the present invention provides compounds useful for th e treatment of microbial infections and/or disorders relating to a microbial infection. It will be appreciated that the compounds of the present invention can exist in free form for treatment, or where appropriate, as a pharmaceutically acceptable derivative thereof. Additionally, it will be appreciated that one or more of the inventive compounds can be formulated with a pharmaceutically acceptable carrier or excipient to provide a pharmaceutical composition.

The composition may be prepared in various forms for administration, including tablets, caplets, pills or dragees, or can be filled in suitable containers, such as capsules, or, in the case of suspensions, filled into bottles. As used herein, “pharmaceutically acceptable carrier medium” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-microbial compounds of the invention, such as by producing any undesirable biological effect or otherwise interacting in a deleterious manner with any other component(s) of the pharmaceutical composition, its use is contemplated to be within the scope of this invention. In the pharmaceutical compositions of the invention, the active agent may be present in an amount of at least 0.1% and not more than 50% by weight based on the total weight of the composition, including carrier medium and/or auxiliary agent(s). Preferably, the proportion of active agent varies between 0.1 to 5% by weight of the composition. Pharmaceutical organic or inorganic solid or liquid carrier media suitable for enteral or parenteral administration can be used to make up the composition. Gelatine, lactose, starch, magnesium, stearate, talc, vegetable and animal fats and oils, gum, polyalkylene glycol, or other known carriers for medicaments may all be suitable as carrier media.

The compounds of the invention may be administered using any amount and any route of administration effective for attenuating infectivity of the microorganism. Thus, the expression “amount effective to attenuate infectivity of a microorganism”, as used herein, refers to a nontoxic but sufficient amount of the anti-microbial agent to provide the desired treatment of microbial infection. The exact amount required will vary from subject to subject, depending on the species, age, and general condition of the subject, the severity of the infection, the particular anti-microbial agent, its mode of administration, and the like. The anti-microbial compounds of the invention are preferably formulated in dosage unit form for ease of administration and uniformity of dosage. The expression “dosage unit form” as used herein refers to a physically discrete unit of anti-microbial agent appropriate for the patient to be treated.

Each dosage should contain the quantity of active material calculated to produce the desired therapeutic effect either as such, or in association with the selected pharmaceutical carrier medium. Typically, the anti-microbial compounds of the invention will be administered in dosage units containing from about 5 mg to about 500 mg of the anti-microbial agent with a range of about 0.1 mg to about 50 mg being preferred.

The compounds of the invention may be administered orally, parenterally, such as by intramuscular injection, intraperitoneal injection, aerosol, intravenous infusion or the like, depending on the severity of the infection being treated. The compounds of the invention may be administered orally or parenterally at dosage levels of about 0.1 mg/kg to about 50 mg/kg and preferably from about 2 mg/kg to about 25 mg/kg, of patient body weight per day, one or more times a day, to obtain the desired therapeutic effect.

According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, means a non-toxic, inert solid, semi-solid or liquid filler, diluent, encapsulating material or formulation auxiliary of any type. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally, intracistemally, intravaginally, intraperitoneally, topically (as by powders, ointments, or drops), bucally, or as an oral or nasal spray.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Uses of Compounds and Pharmaceutical Compositions

According to the methods of treatment of the present invention, microbial infections are treated or prevented in a patient or organism such as a human, lower mammal, fish, bird, or other organism, by administering to the patient a therapeutically effective amount of a compound or pharmaceutical composition of the invention, in such amounts and for such time as is necessary to achieve the desired result. In certain preferred embodiments, the compounds of the present invention are capable of acting as broad spectrum antibiotics and are effective against Gram-negative bacteria. By a “therapeutically effective amount” of a compound of the invention is meant a sufficient amount of the compound to treat microbial, e.g., bacterial infections, at a reasonable benefit/risk ratio applicable to any medical treatment. It will be understood, however, that the total daily usage of the compounds and compositions of the present invention will be decided by the attending physician within the scope of sound medical judgment.

As discussed above and as exemplified in greater detail below, the compounds of the present invention are useful as anti-microbial agents, and thus may be useful in the treatment or prevention of microbial infections. As used herein, unless otherwise indicated, the terms or phrases “microbial infection” and “disorder relate to a microbial infection” include, but are not limited to, infection by the following, bacterial, fungi, yeast, or protozoa.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another antibiotic), or they may achieve different effects (for example, surgery for removal of a tumor, administered concurrently with an inventive antibiotic).

In but one example of the usefulness of combination therapy, it has been shown that treatment with an antibiotic appears to have protective effects against atherosclerosis complications. Specifically, it has been shown that infection with Chlamydia pneumoniae is a contributing factor in the pathogenesis of atherosclerosis (Movahed, M. R. J.S.C. Med. Assoc. 1999, 95, 303). C. pneumoniae and its constituents, such as specific antigens and even DNA, have been detected in atherosclerotic plaques and also in endothelium, smooth muscle cells, and macrophages of arterial walls with atherosclerosis, but have not been found in normal arteries. Thus, treatment with an antibiotic may be used in combination with other therapies, such as surgery or other medication, to more effectively mitigate the symptoms of this disorder.

In yet another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance that can be adapted to the practice of this invention in its various embodiments and the equivalents thereof.

### EXAMPLES

### EXAMPLES

### Example I

### Identifying FtsZ Inhibitors

This example describes the identification of small molecule inhibitors on the activity of the bacterial cell division protein, FtsZ.

Initial Library Screening: Inhibition of FtsZ Activity in vitro

NADH Assay

An enzyme-coupled assay for assembly-dependent FtsZ GTPase activity was developed. Purified FtsZ protein is combined in a reaction vessel with the enzymes, pyruvate kinase, and lactate dehydrogenase, and the substrates GTP, PEP, and NADH. As shown in FIG. 7, upon reaction with GTP, FtsZ yields the products GDP and phosphate, providing a substrate, GDP, for pyruvate kinase in combination with PEP to generate pyruvate. Pyruvate in turn becomes a substrate for lactate dehydrogenase with NADH to yield NAD⁺ and lactate. Test molecules may be added to the reaction mixture to assess their effect on FtsZ activity. Activation of FtsZ activity can be determined by measuring a decrease in the rate of NADH fluorescence compared to the absence of the test molecule (excitation: 355 nm, emission: 460). This assay was miniaturized by testing compounds in a multi-well plate and assessing fluorescence using a Wallac plate reader.

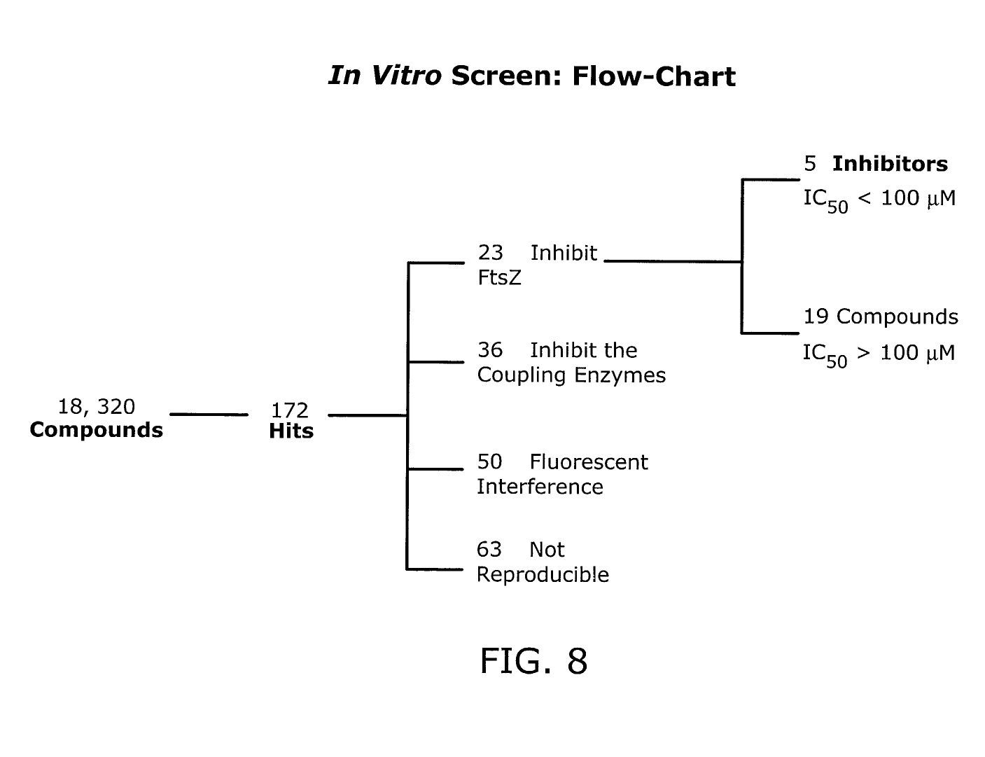

This assay was miniaturized for high throughput screening of the Chembridge 16,320-member small molecule library and the ˜2000 member NCI mini diversity library against untagged, assembly-competent FtsZ purified from Escherichia coli. The MDS1 library was also screened, but no downstream validation assay could be carried out due to lack of compound, so none of the inhibitors identified are from the MDS1 library. From this screen, the in vitro assay identified 172 compounds. From this assay 23 compounds were identified that inhibited FtsZ activity. In a secondary screen, five inhibitors were verified (see FIG. 8).

As depicted in FIGS. 3 and 6, five inhibitors (58-P18 (NCI library), 16-L09 (Chembridge library), 18-M04, 27-D12 (Chembridge library), and 27-F02 (Chembridge library)) and of FtsZ were identified in these initial screens. A phenotypic screen using AcrAB efflux pump knockout strains identified 26E-10.

Downstream Assays Used for Validation

TLC Assay

As noted above, several downstream assays were developed to validate the primary hits, including the NADH assay, described above. Another such assay is the radioactive thin-layer chromatographic (TLC) analysis that measures the conversion of [α-³²P]GTP to [α-³²P]GDP catalyzed by FtsZ. In this assay, the two nucleotides (GTP and GDP) are present in reaction aliquots and are separated on a polyethyleneimine-cellulose thin-layer plate. This affords rapid and direct estimation of the GTPase activity of FtsZ under reaction conditions that are known to promote FtsZ assembly. The reaction mixture does not contain other enzymes or substrates unlike the situation with the enzyme-coupled assay used for primary screening. Therefore, the TLC assay result is a reliable indicator of the in vitro efficacy of the candidate molecules identified as hits using the coupled assay.

TLC analysis of FtsZ GTPase activity in the presence or absence of test compounds was also conducted. 5 μM FtsZ was incubated with 1 mM [α-³²P]GTP (1.5 μCi) at 30° C. and 2 μl aliquots were withdrawn at 5, 15, and 30 min intervals into an equal volume of 1% SDS-20 mM EDTA to quench the reaction. The aliquots were incubated at 70° C. for 2 minutes prior to spotting 0.5 μl samples on a PEI-cellulose plate. The TLC plate was developed in 0.75 M potassium phosphate buffer (pH 3.4), air-dried, and exposed to a film or a phosphorimager.

Inhibition of FtsZ on Ring Assembly and Cell Division in E. coli

The effect of the identified small molecule inhibitor 26E-10 on FtsZ ring assembly was tested by employing a single-copy ftsZ-gfp fusion construct that was integrated at the phage lambda attachment site on the E. coli chromosome. It should be noted that the wild-type, untagged ftsZ gene is also present on the chromosome at its normal locus. The expression of the fusion gene was placed under the control of a mutationally weakened, IPTG inducible tac promoter. Since the level of FtsZ protein expression in the cell is critical for proper cell division, the fusion gene was expressed from a single-copy and at the lowest possible inducer concentration to generate a low level of fluorescently tagged FtsZ-GFP, which did not cause any noticeable cell division aberrations. However, this low level expression was sufficient for imaging the in vivo assembly of the FtsZ-GFP fusion protein by fluorescence microscopy.

We found that the compound-induced phenotype is more pronounced in an acrAB deletion background, suggesting the likely involvement of the drug pump in reducing the intracellular concentration of compounds such as 26E-10. Therefore, in order to test the effect of 26E-10, a culture of the E. coli K-12 strain MC 1000 containing acrAB deletion (henceforth designated as strain DRC 39) was grown.

To an early log-phase culture of DRC 39 (˜1-2×10⁸/ml), 26E-10 was added at its MIC (minimum inhibitory concentration) of 10 μM. Aliquots were withdrawn at 15, 30, 60, and 90 min intervals after the addition of the compound and the cells in the aliquots were fixed immediately with a mixture of glutaraldehyde and para-formaldehyde. This fixation step was carried out to ensure that the handling of cells before microscopy did not cause any artifactual destabilization of the FtsZ ring structure. The fixed cells were washed with PBS, stained with DAPI to visualize the nucleoids, and observed under a fluorescence microscope. In some experiments, the cells were embedded on a thin layer of agarose before microscopy for ease of visualization. After examining the cells for GFP (FITC filter set) and DAPI (DAPI filter set) fluorescence, the DIC digital images of cell morphology were recorded using Nomarski optics (differential interference contrast, DIC, microscopy). Images were taken using an Olympus fluorescence microscope equipped with a CCD camera. Images were finally imported into Adobe Photoshop for processing and presentation.

Those skilled in the art will appreciate that MIC determinations for the compounds identified are made for a wide range of bacteria, including BW pathogens such as Bacillus anthracis, to determine which compounds have a broad-spectrum effect.

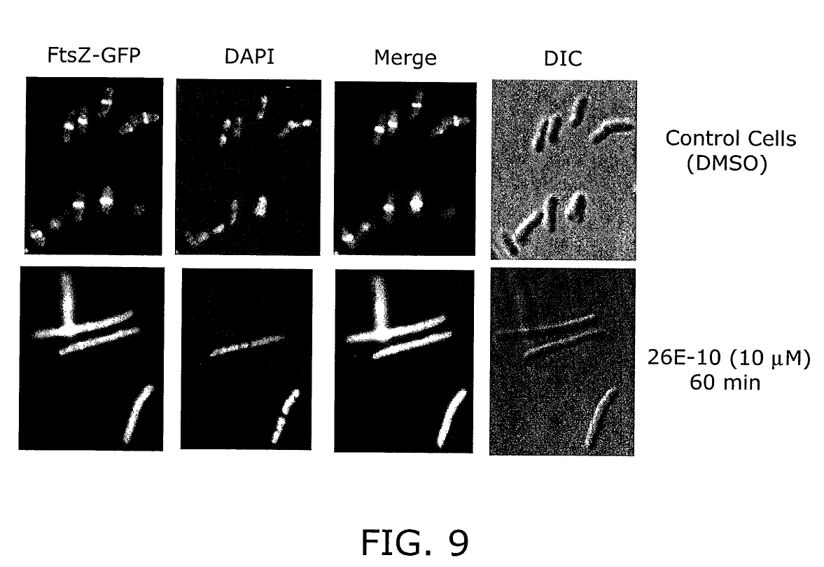

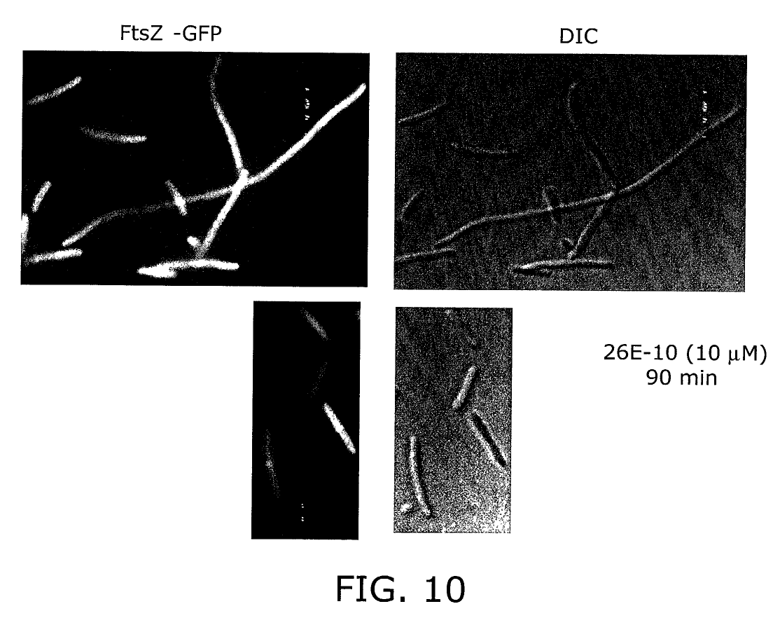

As shown in FIG. 9, the control culture (no 26E-10 added) displayed a distinct equatorial FtsZ-GFP ring structure (Z-ring) at the center of the cells. DAPI staining revealed that the cells carrying Z-rings had segregated nucleoids present on either side of the ring. However, upon treatment with 26E-10, DRC 39 cells started filamenting (˜4× long cells seen within 60 min of treatment), which is indicative of a defect in cell division. Concurrently, the Z-rings appeared markedly reduced in number and diminished in intensity in these elongated cells, with appreciable GFP fluorescence distributed along the length of the cell body. This suggests that 26E-10 is inhibiting de novo Z-ring assembly, perhaps by destabilizing the ring structures.

It is important to note that the fluorescence intensity in the treated cells was significantly higher compared to the control culture and the exposure time for imaging the treated cells was 5-10 fold lower than that for the control cells. It is known that aberrant polymerization of FtsZ-GFP fusion in the cell cytoplasm causes the emitted GFP fluorescence to be intense. Even though we could not discern any such aberration under the microscope, it is possible that 26E-10 may induce inappropriate polymers to form.

We would also point out that DAPI staining showed that the filamenting cells contained mostly regularly spaced nucleoids, suggesting that 26E-10 does not affect DNA replication or chromosome segregation. However, DAPI staining of the elongated cells was not uniform because the cells were fixed but not permeabilized. To avoid this problem, blue Hoechst 33342 dye, which efficiently stains nucleoids in unpermeabilized E. coli cells, was used.

FIG. 10 shows two fields of the phenotype of a 26E-10 treated culture. Images were captured as described above. After 90 min treatment: there was a mixture of 1× to 8× long cells and most of the cells were devoid of distinct Z-rings irrespective of their age as evident from the cell length distribution. This indicates that 26E-10 is targeting Z-ring assembly and the effect increases in severity with the time of treatment. The fact that there were 1× cells in the culture indicates that cell division was continuing at a low level presumably because Z-rings were stochastically forming in some cells in the presence of 26E-10 and some of these rings could complete the septation process.

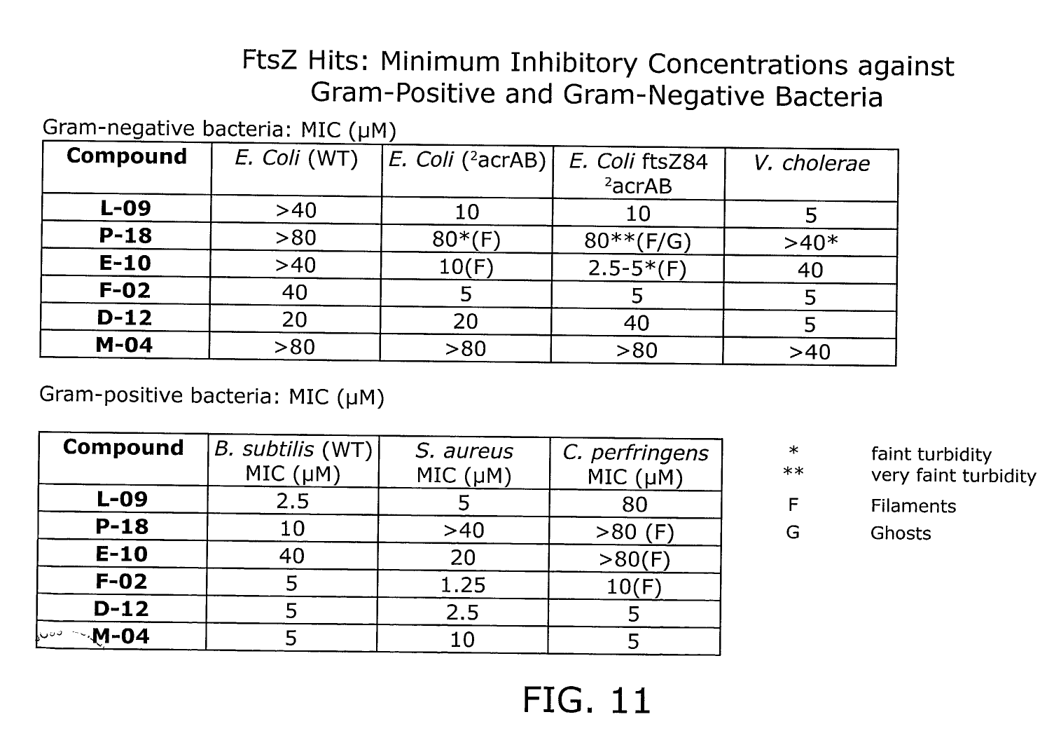

The in vivo effects of the compounds were also tested in a number of other microbial cells, including E. coli ΔacrB::kan, Hemophilus influenzae, Staphylococcus aureus, and Vibrio cholerae, as shown in FIG. 11. Specifically, a cell culture was grown up and diluted 1:5000 (10⁵ to 5×10⁵ cells/ml) as a starter inoculum. Thereafter, one of the identified test compounds was added at concentrations ranging from 1.25 μM to 40-80 μM in DMSO. The samples were incubated at 37° C. and aerated on a rotary wheel for 16 hours and the level of growth assessed by the turbidity of the culture visually. The data in FIG. 11 represents the minimum inhibitory concentration (MIC) of compound that was required to completely inhibit growth of the bacterial culture.

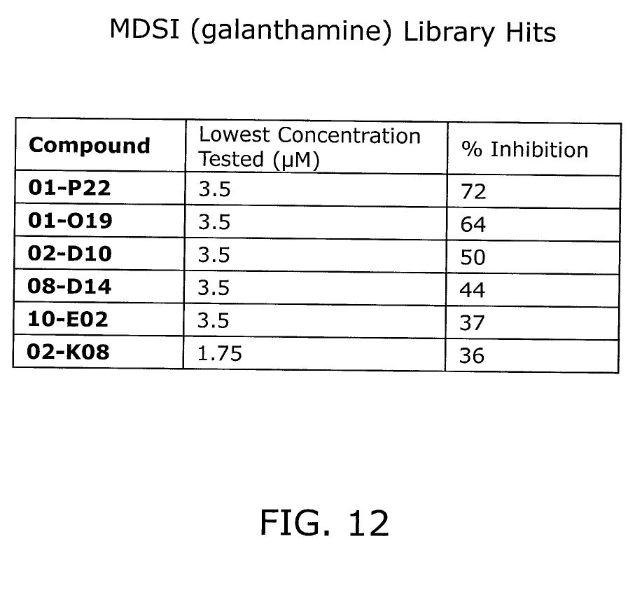

Additional compounds were preliminarily identified from the MDS1 galanthamine library. FIG. 12 depicts the percent inhibition measured by the NADH assay, described above. In some cases positive identification of a compound was validated using the GTPase assay with charcoal, described above. These studies confirmed that low, levels of FtsZ inhibitors affect FtsZ function.

Genetic Evidence of FtsZ Inhibition in vivo

Results presented in FIGS. 10, 11, and 12 provide the cell biology perspective on the effect of 26E-10 in E. coli cells. In order to understand whether 26E-10 indeed specifically targets FtsZ in vivo, a simple experiment was devised using the well-characterized thermosensitive ftsZ84 mutant of E. coli. This mutant is conditional-lethal because it grows and divides at 30° C., but undergoes a cell division block at 42° C. that leads to lethal cell filamentation. The division block of the ftsZ84 mutant at 42° C. is due to a drastic destabilization of the Z-rings at high temperature (within a minute after temperature shift-up). Based on the theory that 26E-10 was inhibiting the in vivo assembly of FtsZ, we reasoned that in the presence of 26E-10, the mutant Z-ring may not be as robust as the wild-type ring at the permissive temperature of 30° C. and this inherent weakness of the mutant ring may be exacerbated.

We assessed the sensitivity of the ftsZ84 mutant strain DRC40 (harboring the plasmid vector pBR322) to 26E-10 and 27F02 at 30° C. in comparison to the congenic parent DRC39. Both DRC39 and DRC40 lack the major multidrug efflux pump AcrAB.

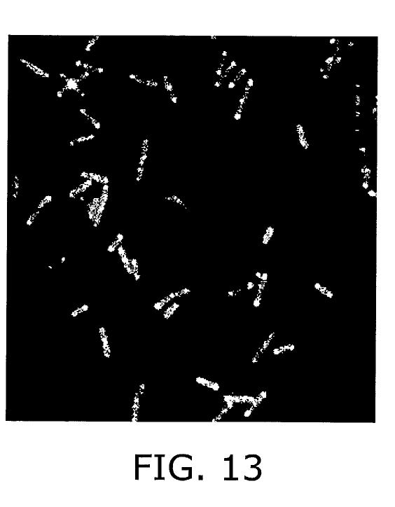

DRC39 cells were treated for 2 hours with inhibitor 27F02. It was found initially that 27F02 kills cells, but no filamentation phenotype was observed (see FIG. 13). The high fluorescent background of 27F02 itself was precluding FtsZ-GFP ring imaging or FtsZ ring imaging using immunofluorescence. Viewing the effect of 27F02 on FtsZ ring structure in vivo in DRC39 cells (using the RITC filter set for GFP imaging), the majority of cells have distinct bipolar fluorescent foci and infrequently a central focus that did not span the entire circumference of the cell. (see FIG. 13).

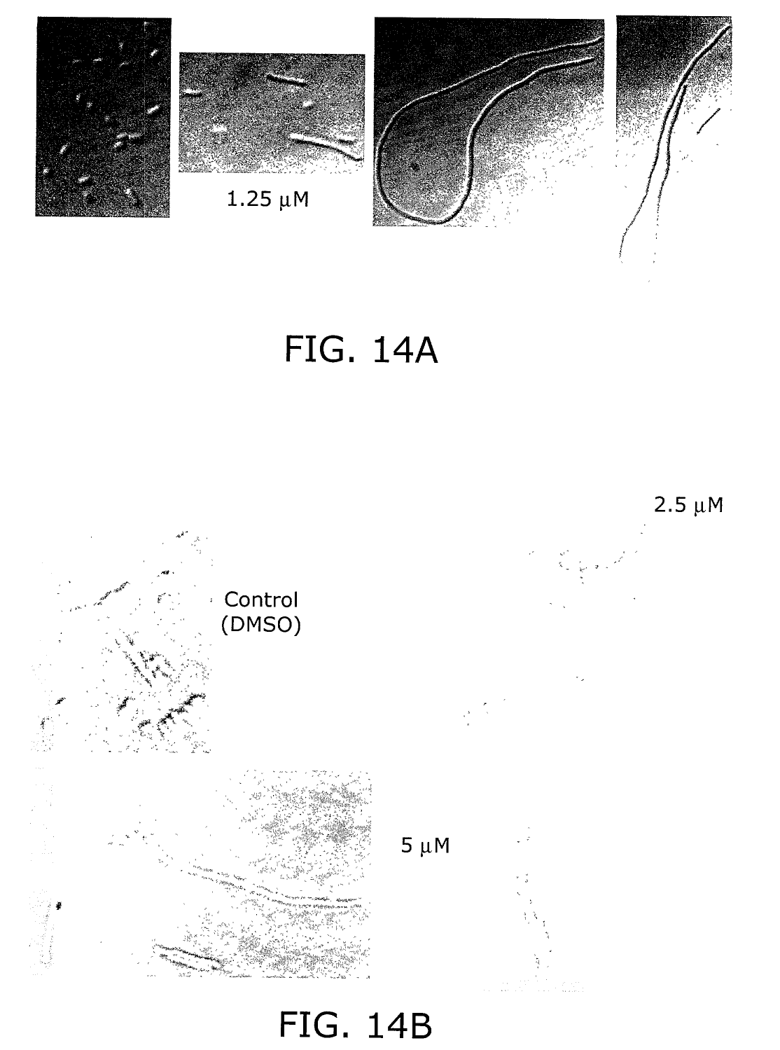

We interpret this result as 27F02 interacting in vivo with division ring components such as FtsZ. The polar foci may be remnants of old division rings that did not disassemble completely. Alternatively, nascent ring machinery may be assembling inappropriately at the poles. Most strikingly, the majority of cells lack a distinct central ring, suggesting inhibition of medial FtsZ ring assembly. Moreover, the partial ring-like foci at midcell are also indicative of aberrant assembly or destabilization of division rings in the presence of 27F02.

Whereas the MIC of 26E-10 for DRC39 is 10 μM, it was between 2.5 to 5 μM for DRC40. The DRC40 cell density at 2.5 μM compound was very low compared to the untreated control, with predominantly long filaments (16×), filamentous ghosts, and few short cells present (FIG. 14). DRC40 showed absolutely no growth at 5 μM, whereas DRC39 had a mixture of filaments of varying lengths and short cells present at a low density at this concentration of 26E-10. The cell density of DRC39 at 5 μM was appreciably higher than that of DRC40 at 2.5 μM. These results indicate that the ftsZ84 mutant (DRC40) has higher sensitivity toward 26E-10 compared to its wild-type parent (DRC39), likely because the presence of 26E-10 augments the inherent weakness of the ftsZ84 ring structure, akin to a synthetic lethal genetic interaction.

Whether the higher sensitivity of ftsZ84 to 26E-10 could be reversed in the presence of the recombinant pBR322 plasmid carrying the wild-type ftsZ gene was also tested. Indeed, when wild-type ftsZ was provided in trans, the MIC of 26E-10 for the mutant DRC40 was 10 μM, identical to that seen with the DRC39 parental cells. As shown in FIG. 14B, DRC40/pBR-ftsZ cells underwent robust cell division at 2.5 μM 26E-10 unlike the situation with DRC40/pBR322. Moreover, at 5 μM 26E-10, there was higher cell density and less pronounced filamentation with DRC40/pBR-ftsZ⁺ in contrast to DRC40/pBR322 (compare FIGS. 14A and 14B). These results provide compelling genetic evidence that 26E-10 targets FtsZ rings in vivo.

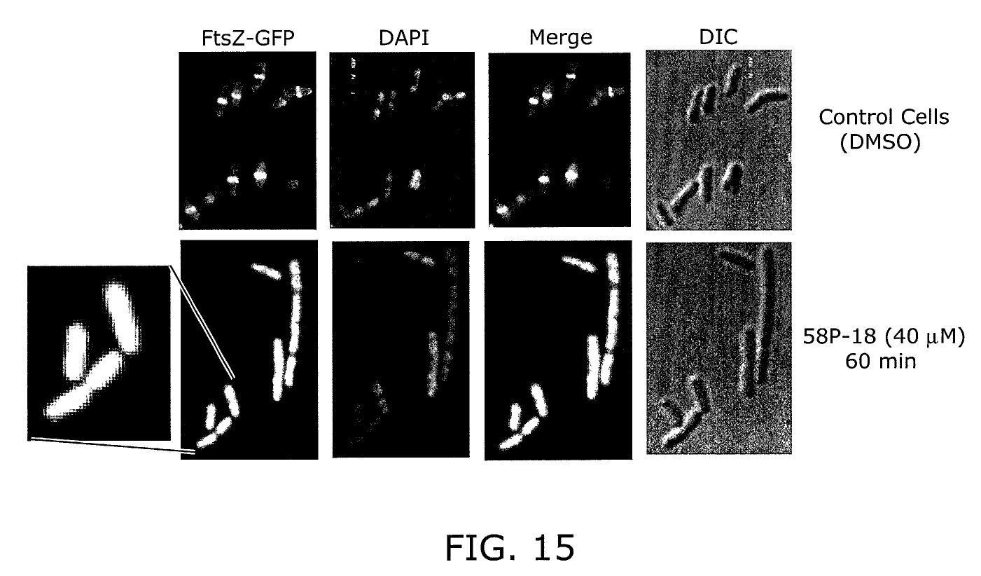

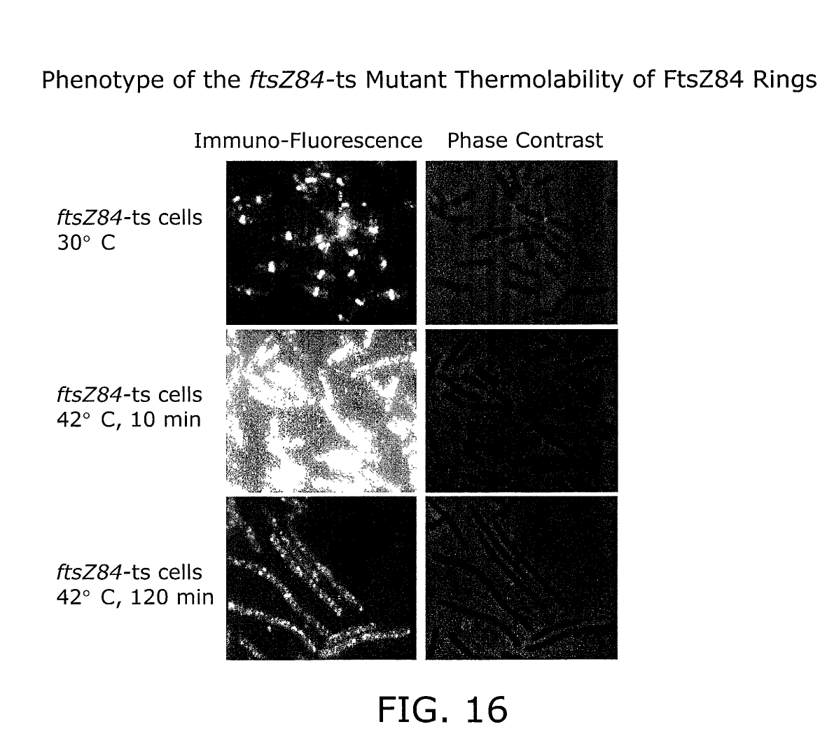

In addition, the phenotype of ftsZ84 cells was examined in the presence of 26E-10 over time to determine the thermolability of the ftsZ84 rings. Specifically, the phenotype of the thermosensitive ftsZ84 was assessed at 30° C., and also at 42° C. at 10 and 120 minutes. At 42° C. the mutant FtsZ rings were rapidly destabilized, within 10 minutes. A similar phenotype is expected with small molecules that inhibit or activate polymerization-dependent FtsZ GTPase activity. (See FIG. 15).

ZipA interacts with FtsZ both in vitro and in vivo and it has been shown that a second copy of ZipA can suppress the thermosensitivity of the ftsZ84 mutant at the restrictive temperature of 42° C. This is because ZipA is a stabilizer of FtsZ ring assembly and doubling the number of ZipA molecules in the cell leads to the stabilization of the thermolabile FtsZ84 ring in vivo.

A new result is that a second copy of the essential division gene zipA can also decrease or ameliorate the toxicity of 26E-10. The fact that a second copy of zipA can reverse the toxicity of 26E-10 to a significant degree suggests that 26E-10 may be destabilizing the FtsZ ring structure in vivo.

Effect of an in vitro Inhibitor of FtsZ GTPase in vivo

The effect of 58P-18 on Z-ring assembly and cell division was tested in DRC39 cells in a similar manner as that described above for 26E-10. FIG. 16 shows that cells start elongating within 60 min of treatment with 40 μM 58P-18 and, more strikingly, none of the treated cells appear to contain a medial FtsZ ring. Even though DAPI staining shows that the nucleoids have replicated and segregated in the presence of the compound, none of the short cells in the field has been able to assemble a Z-ring between segregated nucleoids. Instead, the GFP fluorescence is delocalized all over the cell body (fluorescence is much more intense than in the control cells, similar to that seen with 26E-10), suggesting the possibility of aberrant FtsZ-GFP polymerization. Interestingly, in contrast to 26E-10, treatment with 58P-18 seems to generate a pattern of FtsZ-GFP distribution in cells that is very similar to that seen with DAPI stained nucleoids. Thus, without limiting the mechanism of the invention, 58P-18 may promote inappropriate association of FtsZ with the chromosome.

Confirmation of FtsZ Inhibitor Activity

A high throughput enzyme-coupled FtsZ assay is used to preliminarily screen the combinatorial libraries, synthesized in the Candidate inhibitors of FtsZ GTPase activity identified in the FtsZ assay and are then tested in the downstream charcoal-based GTPase assay, also described above. The inhibitory activity of the compounds are tested at two concentrations (e.g., 3.5 μM or 17.5 μM) relative to the DMSO-only control in the downstream charcoal-based assay for GTPase activity described above.

Phenotypic Screen: in vivo Assay for Inhibition of Bacterial Growth

Compound Libraries are further screened in an in vivo assay for bacterial growth. An overnight culture of the wild-type E. coli lacking the major drug efflux pump AcrAB (MC1000 ΔacrAB=DRC 39) is diluted to 1:5000 in fresh medium and 40 μl is inoculated into each well of a clear bottom NUNC 384-well plate. 100 nL of compounds is pin transferred to each well using the Cartesian robot, resulting in a final screening concentration of 17 μM. The 384-well plates are incubated at 37° C. in a humid chamber and the culture turbidity is measured at 650 nm using the Wallac Plate Reader after 5 h and after 24 h.

The reduction in cell density in the presence of a compound after 24 h or 48 h is expressed as the standard deviation from the average final density of the other wells on the plate. The effect of the compounds is also expressed as percent growth inhibition compared to the DMSO only control. A compound was characterized as a “hit” if it caused the cell density in a well to decrease by two standard deviations from the mean density of all wells in the plate. Samples from the growth-inhibited wells are then visualized by DIC microcopy and inspected for filaments or mini cells, phenotypic markers for cell division aberrations.

Screening for FtsZ Inhibitors Using Small Molecule Microarrays

The MDS1 library is printed on glass slides to create small molecule microarrays to provide an opportunity to explore the feasibility of using such microarrays to identify FtsZ antagonists. The microarray is created by using a high-precision robot to pick up a small volume of dissolved compounds from the original 384 well plates and repetitively deliver 1 nL of solution to defined locations on a chemically derivatized glass microscope slide. Each compound is immobilized on the glass slide via a covalent linkage between a common functional group on the small molecule and the maleimide-derivatized glass slides. Interactions between FtsZ and small molecules are determined by incubating the microarray slide with purified FtsZ-GFP fusion protein and then visualizing the location of the bound protein by the ArrayWoRx fluorescent slide scanner. This experiment is performed in the absence of GTP to identify compounds that bind FtsZ monomers and in the presence of GTP to identify compounds that bind FtsZ polymers. Data obtained from screening the microarray library may validate the initials hits identified in the enzyme-coupled biochemical screen and provide evidence for the utility of small molecule microarrays as a fast and efficient method for screening future chemical libraries.

Screening a chemical library in a microarray format improves the speed of the screening method and also increases the reliability of the assay by comparing the hits identified by microarray analysis with those obtained from other in vitro and cell-based screening assays. In addition, the combination of the microarray assay with other assay methods will also assist in the validation of the targets identified, e.g., by comparing the targets identified in one assay to the targets identified in the other assay. Validation of inhibiting and activating structures is important for molecular modeling and generation of more potent derivatives against a given target.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A method of treating a microbial infection, comprising:
providing a pharmaceutical composition containing an inhibitor of FtsZ; and
administering the composition to a patient in need thereof.

2. A method of detecting FtsZ activity, comprising steps of:
combining FtsZ pyruvate kinase, lactate dehydrogenase, GTP, PEP, and NADH in a reaction mixture; and
detecting a change in the rate of the decrease of NADH fluorescence in the reaction mixture.

3. The method of claim 2, wherein the step of detecting comprises measuring the change in the rate of the decrease of NADH fluorescence uses a fluorescence spectroscopy.

4. The method of claim 2, wherein the reaction mixture is placed in a multi-well plate.

5. The method of claim 4, wherein detecting comprises measuring the change in the rate of the decrease of NADH fluorescence uses a fluorescent plate reader.

6. A method for identifying compounds that affect cell division, comprising steps of:
expressing the FtsZ protein in a cell;
contacting the cell with a compound; and
detecting a defect in cell division.

7. The method of claim 6, wherein the defect in cell division is an activation of cell division.

8. The method of claim 7, wherein the activation of cell division causes a phenotype of minicells devoid of DNA.

9. The method of claim 6, wherein the defect in cell division is an inhibition of cell division.

10. The method of claim 9, wherein the inhibition of cell division causes a phenotype of long filamentous cells.

11. A method of identifying compounds that affect cell division, comprising steps of:
contacting a cell that is defective in cell division with a compound of interest; and
detecting an alteration in the phenotype of the cell.

12. The method of claim 11, wherein the cells are ftsZ84 cells.

13. The method of claim 11, phenotype would be a destabilization in the Z ring structure.

14. The method of claim 11, wherein the phenotype is an exacerbation of the failure to form Z rings.

15. An assay system for identifying compounds that effect cell division, the assay system comprising:
a reaction mixture comprising a FtsZ protein, pyruvate kinase, lactate dehydrogenase, GTP, PEP, and NADH.

16. An assay system for identifying compounds that effect cell division, the assay system comprising:
bacterial cell that has a mutation effecting a multidrug efflux pump, wherein the bacterial cell further expresses a FtsZ protein.

17. The assay system of claim 16, wherein the bacterial cell is a ftsZ84 bacterial cell.

18. A pharmaceutical composition for inhibiting cell division, comprising:
an effective amount of one or more compounds selected from the group consisting of 58-P18, 16-L09, 18-M04, 27-F02, and 26-E10 of FIG. 3 and FIG. 6.
